(19) United States
(12) Patent Application Publication
(10) Pub. No.: US 2023/0332244 A1
(43) Pub. Date: Oct. 19, 2023

(54) METHODS FOR THE RAPID ASSESSMENT OF THE EFFICACY OF CANCER THERAPY AND RELATED APPLICATIONS

(71) Applicant: aiGENE, Inc., Aurora, CO (US)

(72) Inventor: Floyd TAUB, Golden, CO (US)

(21) Appl. No.: 18/162,133

(22) Filed: Jan. 31, 2023

Related U.S. Application Data
(63) Continuation of application No. PCT/US2021/044225, filed on Aug. 2, 2021.
(60) Provisional application No. 63/063,267, filed on Aug. 8, 2020.
(60) Provisional application No. 63/060,004, filed on Aug. 1, 2020.

(51) Int. Cl. C12Q 1/6886; G01N 33/487

(52) U.S. Cl. CPC C12Q 1/6886; G01N 33/48707; C12Q 1/6825; C12Q 2600/156

(57) ABSTRACT

The present invention relates to methods for the rapid assessment of the efficacy of cancer therapies based on the detection of nucleic acid markers early after the onset of said therapies, as well as application of said methods in cancer therapy and clinical trial design. Novel clinical trial designs that allow approval of a drug as part of a plan requiring diagnostic testing while on drug and changing to standard of care within the same experimental arm are presented. They allow approval of a drug as part of a procedure when the drug alone would not be approved.

### CROSS-REFERENCE TO RELATED APPLICATIONS

This application is a continuation application of International Application No. PCT/US2021/044225, filed Aug. 2, 2021, which claims the benefit of priority under 35 U.S.C. §119(e) to U.S. Provisional Pat. Application No. 63/060,004, filed Aug. 1, 2020, and to U.S. Provisional Pat. Application No. 63/063,267, filed Aug. 8, 2020. The entire disclosures of U.S. Provisional Pat. Application Nos. 63/060,004 and 63/063,267 are incorporated herein by reference.

### FIELD OF THE INVENTION

The present invention relates to methods for the rapid assessment of the efficacy of cancer therapies based on the detection of nucleic acid markers early after the onset of said therapies, as well as applications of said methods in cancer therapy and especially in clinical trial design. Novel clinical trial designs that allow approval of a drug as part of a plan requiring diagnostic testing while on drug and changing to standard of care within the same experimental arm are presented. They allow approval of a drug as part of a procedure when the drug alone would not be approved

### BACKGROND

Hyperprogressive disease (HPD) has become a serious clinical problem in the field of cancer therapies. HPD is when a cancer grows dramatically faster as a result of therapy. Depending on the definition of the term “faster”, as well as on the clinical population, it has been reported to occur in up to 29% of patients who are given immune-oncological therapy (IO), although about 20% of patients may be a more accurate estimate. Clinically HPD and progressive disease (PD) both are a sign of drug failure and an indication that the therapy should be changed. One typically the criteria is a doubling in the cancer.

The size of solid cancers is typically measured using imaging techniques such as X-rays, CAT (computer-assisted tomography) scans, MRIs (magnetic resonance imaging), radionuclide imaging or other methods known in the art. In standard clinical practice, however, assuming no special indications, imaging is not done until 2 to 3 months after the start of a cancer therapy. Earlier imaging may miss responses or progression, as imaging is a lagging indicator of the efficacy of therapy. Currently, despite the disadvantages all known methods for diagnosing HPD involve imaging confirmation. These often require two images taken prior to treatment to measure the pre-treatment cancer growth rate. Lack of this or other images or imaging problems can prevent the analysis of patients.

For immunotherapies, imaging is further problematic since a tumor can appear to be larger due to the influx of immune cells, which is actually a sign of the therapy working. Despite this increase in size of the lesion (pseudo-progression), these patients should remain on their immunotherapy. In contrast, those patients with HPD and those patients whose cancer continues to grow despite the immunotherapy should have their therapy changed as early as possible. Both pseudo-progression and slow response makes imaging unreliable as a measure of IO success soon after the therapy is initiated. In addition, imaging may be expensive, requires additional visits, entails additional exposure to the medical environment with attendant infection risks, requires special facilities, may expose the patient to potentially mutagenic radiation, as well as being stressful and inconvenient. Further, in animals such as pets, imaging typically requires anesthesia.

Moreover, it should be noted that many lesions are unmeasurable by imaging, while others, such as bone lesions, do not change in imaging even with effective therapy. Even measurable lesions change shape making estimations of growth or shrinkage problematic. Further, many cancer lesions are not measurable as they are (i) too small, but sum to severe and significant disease, (ii) have ill-defined borders, and/or (iii) are partially obscured by normal or abnormal tissue. In addition, cancers change shape with therapy, making accurate quantitative comparisons difficult. Further, clinicians inaccurately estimate tumor mass (volume) by measuring a single largest diameter, typically on a 2D image.

The same applies to clinical trials for cancer therapy which usually focus on single drugs or fixed combinations of drugs which patients take until an endpoint, or for a fixed period of time. Typically results are compared to the Standard of Care (SOC). The endpoint is typically progressive disease or unacceptable toxicity.

For oncology clinical trials, it has been difficult or impossible in the past to rapidly determine if a new drug is effective in increasing survival. Again, the only broadly accepted surrogate marker which applies to a wide range of cancers is imaging as discussed above. Even then patients are typically kept on the same drug protocol unless they have shown progressive disease, typically defined by RECIST 1.1 (Response Evaluation Criteria In Solid Tumors) or irRECIST (immune response RECIST). Even classic cytotoxic drugs may have an immune component to their activity and thus are in part IO however classic not immune response RECIST are used for this type of drug.

Accordingly, the technical problem underlying one aspect of the present invention is the provision of means for the rapid assessment of the efficacy of cancer therapies based on the detection of nucleic acid markers early after the onset of said therapies, as well as applications of said methods in cancer therapy and clinical trial design.

### SUMMARY

One embodiment relates to a method of performing a clinical trial for a cancer therapy regimen, comprising the steps of (a) administering a first cancer therapy regimen to a first group of patients having a cancer; (b) determining the therapeutic efficacy of said first cancer therapy regimen for patients in the first group of patients by a method, comprising measuring a nucleic acid marker of cancer burden in a biological sample from the patients; wherein the first cancer therapy regimen is identified as being efficacious in a patient when the nucleic acid marker indicates a lower cancer burden or a stable cancer burden as compared to (i) one or more prior measurements of the nucleic acid marker in that patient after onset of the first cancer therapy regimen, or (ii) one or more measurements of said nucleic acid marker in that patient prior to onset of the first cancer therapy regimen, and wherein the first cancer therapy regimen is identified as not being efficacious in a patient when the nucleic acid marker indicates a higher cancer burden as compared to (iii) one or more prior measurements of the nucleic acid marker in that patient after onset of said cancer therapy regimen, or (iv) one ore more measurements of the nucleic acid marker in that patient prior to onset of the first cancer therapy regimen; and (c) continuing to administer the first cancer therapy regimen to a patient when the first cancer therapy regimen is identified as being efficacious in that patient, and discontinuing the first cancer therapy regimen and beginning to administer a second cancer therapy regimen to a patient when the first cancer therapy regimen is identified as not being efficacious in that patient, but retaining the patients receiving the second cancer therapy regimen in the first group of patients.

In one aspect of the method of performing a clinical trial, the clinical trial further comprises administering a standard of care (SOC) therapy regimen to a control group of patients having the cancer and comparing the above embodiment to this SOC group.

Another embodiment relates to a method of performing an early stage clinical trial comprising the steps of (a) administering a first dose level of a first cancer therapy regimen to a first group of patients; (b) determining the therapeutic efficacy of the dose level for patients in the first group of patients by a method, comprising measuring a nucleic acid marker of cancer burden in a biological sample from the patients; wherein the dose level is identified as being efficacious in a patient when the nucleic acid marker indicates a lower cancer burden or a stable cancer burden as compared to (i) one or more prior measurements of the nucleic acid marker in that patient after onset of the first cancer therapy regimen or (ii) one or more measurements of said nucleic acid marker in that patient prior to onset of the first cancer therapy regimen, and wherein the dose level is identified as not being efficacious in a patient when the nucleic acid marker indicates a higher cancer burden as compared to (iii) one or more prior measurements of the nucleic acid marker in that patient after onset of said cancer therapy regimen or (iv) one or more measurements of the nucleic acid marker in that patient prior to onset of the first cancer therapy regimen; and (c) continuing to administer the first dose level to a patient when the first cancer therapy regimen is identified as being efficacious in the patient, and discontinuing the patient from the trial or escalating that patient to a higher dose level if the first dose level is identified as not being efficacious.

Another embodiment relates to a method of evaluating a hospice patient comprising the steps of (a) administering a cancer therapy regimen to a hospice patient; (b) determining the therapeutic efficacy of said cancer therapy regimen in the hospice patient by a method comprising measuring a nucleic acid marker of cancer burden in a biological sample from the patient; wherein the cancer therapy regimen is identified as being efficacious when the nucleic acid marker indicates a lower cancer burden or a stable cancer burden as compared to (i) one or more prior measurements of the nucleic acid marker after onset of the cancer therapy regimen, or (ii) one or more measurements of said nucleic acid marker prior to onset of the cancer therapy regimen, and wherein the cancer therapy regimen is identified as not being efficacious when the nucleic acid marker indicates a higher cancer burden as compared to (iii) one or more prior measurements of the nucleic acid marker after onset of said cancer therapy regimen, or (iv) one or more measurements of the nucleic acid marker prior to onset of the first cancer therapy regimen; and (c) continuing to administer the cancer therapy regimen to the patient when the cancer therapy regimen is identified as being efficacious in the patient, and discontinuing the cancer therapy regimen when the cancer therapy regimen is identified as not being efficacious in the patient.

Yet another embodiment relates to a method of evaluating a cancer patient to be given a cancer drug therapy that might cause hyperprogression comprising the steps of: (a) determining the rate of growth of the patient’s cancer by performing at least 2 measurements of circulating DNA prior to beginning of the therapy; (b) determining the rate of growth of the patient’s cancer by performing at least 1 measurement of circulating DNA after beginning the therapy; and (c) continuing to administer the therapy if the rate of growth has not increased, or discontinuing the therapy if the growth rate has increased.

In any of the embodiments described above or elsewhere herein, the first cancer therapy regimen is selected from the group consisting of immunotherapy, therapy using an antibody, adoptive T cell therapy, chimeric antigen receptor (CAR) T cell therapy, therapy using an antibody-drug conjugate, a cytokine therapy, therapy using a cancer vaccine, therapy using a checkpoint inhibitor, radiation therapy, surgery, therapy using a chemotherapeutic agent, a therapy using a targeted therapy, a therapy using an enzyme inhibitor and combinations thereof.

In any of the embodiments described above or elsewhere herein, the biological sample is selected from the group consisting of whole blood, blood plasma, blood serum, saliva, urine, cerebrospinal fluid, sputum, broncho-alveolar lavage, bile, stool, pleural effusion, lymphatic fluid, cyst fluid, stool, uterine lavage, vaginal fluids, ascites, and combinations thereof.

In any of the embodiments described above or elsewhere herein, the biological sample is obtained from the patients within 1 hour, within 2 hours, within 3 hours, within 4 hours, within 5 hours, within 6 hours, within 8 hours, within 10 hours, within 12 hours, within 15 hours, within 18 hours, within 21 hours, within 1 day, within 2 days, within 3 days, within 4 days, within 5 days, within 6 day, within 7 days, within 8 days, within 9 days, within 10 days, within 11 days, within 12 days, within 13 days, within 14 days, within 3 weeks, within 4 weeks, within 5 weeks, within 6 weeks, within 7 weeks, or within 8 weeks after onset of the first cancer therapy regimen or after the a previous sample was obtained.

In any of the embodiments described above or elsewhere herein, one or more biological samples are obtained from the patients prior to the onset of the first cancer therapy regimen.

In any of the embodiments described above or elsewhere herein, steps (b) and (c) (i.e., steps of determining the therapeutic efficacy (step b) and administering the first cancer therapy or second cancer therapy (step c)) are repeated for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 times during the first cancer therapy regimen.

In any of the embodiments described above or elsewhere herein, steps (b) and (c) (i.e., steps of determining the therapeutic efficacy (step b) and administering the first cancer therapy or second cancer therapy (step c)) are repeated every day, every other day, every third day, twice a week, weekly, every 10 to 14 days, every two weeks, every three weeks, every four weeks or every eight weeks during the first cancer therapy regimen.

In any of the embodiments described above or elsewhere herein, the first cancer therapy regimen is identified as being efficacious when two or more measurements of the nucleic acid marker do not indicate a higher cancer burden or higher growth rate of cancer burden, and wherein the first cancer therapy regimen is identified as not being efficacious when two or more measurements of the nucleic acid marker indicate a higher cancer burden or higher growth rate of cancer burden. In one aspect, a higher cancer burden or higher growth of cancer burden is determined to be when the nucleic acid marker is at least 50%, 40%, 30%, 20%, 10%, or 5%, higher than a measurement of (i), (ii), (iii) or (iv) (i.e., one or more prior measurements of the nucleic acid marker in that patient after the onset of the first cancer therapy regimen ((i) and/or (iii)) or one or more measurements of said nucleic acid marker in that patient prior to the onset of the first cancer therapy regimen ((ii) and/or (iv))).

In any of the embodiments described above or elsewhere herein, the identification of the first cancer therapy as being efficacious or not efficacious is based on a trend or a change in trend of two or more measurements of the nucleic acid marker.

In any of the embodiments described above or elsewhere herein, the patients have a cancer selected from the group consisting of a head and neck cancer, a central nervous system cancer, a lung cancer, a bronchial cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a kidney cancer, a renal pelvis cancer, a bladder cancer, a uterine cancer, a cervical cancer, a thyroid cancer, a muscle cancer, an endocrine cancer, a lymphoma, a bone marrow cancer, a leukemia, a myeloid dysplasia, an epithelial cancer, a cancer derived from endodermal tissue, a cancer derived from ectodermal tissue, a cancer derived from mesodermal tissue, and a prostate cancer.

In any of the embodiments described above or elsewhere herein, a first cancer therapy regimen identified as not being efficacious allows cancer progression or cancer progression at an increased rate in a patient or allows cancer hyperprogression in a patient.

In any of the embodiments described above or elsewhere herein, the nucleic acid marker is a nucleic acid marker having a short half-life. In one aspect, the half-life of the nucleic acid marker is less than 48 hours, less than 24 hours, less than 12 hours, or less than 6 hours.

In any of the embodiments described above or elsewhere herein, the nucleic acid marker is a DNA marker. In one aspect, the DNA marker is a circulating or cell free short DNA marker. In still another aspect, the circulating short DNA marker is a circulating tumor DNA (ctDNA) marker. In yet another aspect, the DNA marker comprises a methylation status of said DNA. In still another aspect, the DNA marker comprises size distribution of said DNA. In yet another aspect, the DNA maker comprises DNA sequence information. In still another, the DNA marker does not comprise DNA sequence information.

In any of the embodiments described above or elsewhere herein, measuring the nucleic acid marker in the biological sample in step (b) (i.e., step of determining the therapeutic efficacy) comprises using an electrode. In one aspect, the electrode is a gold electrode or a graphene electrode. In one aspect, the electrode is a bare gold electrode. In still another aspect, the bare gold electrode is a solid gold electrode, a screen-printed gold electrode, or a thin film gold electrode.

In any of the embodiments described above or elsewhere herein, measuring the nucleic acid marker in the biological sample in step (b) i.e., step of determining the therapeutic efficacy) comprises using electrochemistry. In one aspect, electrochemistry encompasses differential voltammetry, square wave voltammetry, and/or impedance measurements.

In any of the embodiments described above or elsewhere herein, the patients are mammals.

In any of the embodiments described above or elsewhere herein, the first cancer therapy regimen is identified as not being efficacious in a patient, the patient is administered a second cancer therapy regimen. In one aspect, the method further comprising administering a third cancer therapy regimen to a control group of patients having the cancer. In one aspect, the third cancer therapy comprises standard of care therapy for the cancer. In still a further aspect, the second cancer therapy comprises standard of care therapy for the cancer. In still another aspect, the method further comprises comparing the efficacy of treatments received by the first group of patients with the efficacy of treatments received by the control group of patients. In still another aspect, the first group of patients and the control group of patients had the same result on a pre-trial companion diagnostic test.

In any of the embodiments described above or elsewhere herein, the first cancer therapy regimen comprises administration of a drug that failed one or more prior Pivotal trials.

In any of the embodiments described above or elsewhere herein, the first cancer therapy regimen comprises administration of a drug that previously failed a Pivotal Trial and wherein the second regimen includes administration of SOC therapy.

In any of the embodiments described above or elsewhere herein, the first cancer therapy regimen comprises administering a drug that has not been in a Pivotal trial, wherein the drug is the first drug in the first cancer therapy regimen.

### DETAILED DESCRIPTION

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry are those well-known and commonly used in the art.

All publications, patents, and published patent applications referred to in this application are specifically incorporated by reference herein. In case of conflict, the present specification, including its specific definitions, will control.

For people with a significant cancer burden, or a recurrence of cancer, chemo and even targeted therapy prolongs life but only rarely if ever cures the cancer. IO is a revolutionary step in that even some people, generally a group of about 10% with late-stage cancer are cured. However, it does this at the cost of a group of about 20% having hyperprogression and another group having no benefit. The inventors have determined that it can be determined early on which group a patient is in and thus adjust therapy. Different patients benefit from standard of care (SOC) typically chemo than from IO. Methods disclosed herein are used to determine which are which by a brief therapeutic trial of IO.

The classic clinical trial design to approve new cancer drugs (NCD) or regimens or obtain insurance coverage, payment or reimbursement is to compare the fraction of people who will benefit from a new drug to the fraction that benefits from the SOC, median survival is the classical endpoint and imaging is a surrogate endpoint. One aspect of this invention is a novel trial design. It is radically different from previous designs that compare SOC and NCD in that it uses both in a single trial arm. Further it is unique in that it uses cell free DNA (cfDNA) and more specifically circulating tumor DNA (ctDNA) to determine if the NCD drug is working well for each patient and, and while retaining the patient in the experimental arm, switches the patient to SOC if the NCD is not working well. Thus, this arm harvests the benefit of the NCD by identifying patients who actually respond to it plus in this arm patients who do not respond to the NCD get the benefit of SOC. IO has been postulated to potentiate SOC by a process deemed chemosensitization. Chemo based SOC is expected to be especially effective against fast growing HPD.

Based on mathematical modeling and experimental animal work it has been stated that if curing a patient by typical chemo or SOC is very unlikely the typical high dose of such compounds should not be given, instead a dose sufficient to maintain the cancer at a constant minimally symptomatic size should be administered. This steady state approach is dramatically different than the current practice of alternating sequential lines of therapy each designed to kill as much cancer as possible with periods of uncontrolled high growth between the “lines” of therapy, (this high growth postulated to fill in the newly emptied niches). Currently, imaging tools are insufficiently accurate for application of this principle. One aspect of this invention is therapy, in individual patients or in an arm of a clinical trial, could be administered to the level of maintaining at a constant level of cancer by monitoring the ctDNA level. This method is called “Adaptive Therapy”.

One aspect of the invention generally relates to a method of performing a clinical trial for a cancer therapy regimen. The method comprises administering a first cancer therapy regimen to a first group/arm of patients having a cancer; determining the therapeutic efficacy of said first cancer therapy regimen for patients in the first group of patients by a method, comprising a nucleic acid marker of cancer burden in a biological sample from the patients; wherein the first cancer therapy regimen is identified as being efficacious in a patient when the nucleic acid marker indicates a lower cancer burden or a stable cancer burden as compared to (i) one or more prior measurements of the nucleic acid marker in that patient after onset of the first cancer therapy regimen, or (ii) one or more measurements of said nucleic acid marker in that patient prior to onset of the first cancer therapy regimen, and wherein the first cancer therapy regimen is identified as not being efficacious in a patient when the nucleic acid marker indicates a higher cancer burden as compared to (iii) one or more prior measurements of the nucleic acid marker in that patient after onset of said cancer therapy regimen, or (iv) one or more measurements of the nucleic acid marker in that patient prior to onset of the first cancer therapy regimen; and continuing to administer the first cancer therapy regimen to a patient when the first cancer therapy regimen is identified as being efficacious in that patient, and discontinuing the first cancer therapy regimen and beginning to administer a second cancer therapy regimen to a patient when the first cancer therapy regimen is identified as not being efficacious in that patient, but retaining the patients receiving the second cancer therapy regimen in the first group/arm of patients. Reference herein to a first group (or arm) of patients typically refers to an experimental or test group of patients in a clinical trial. That is, a clinical trial arm in which the patients are receiving the experimental treatment. Thus, reference herein to a first cancer therapy typically refers to an experimental treatment such as a new drug. Reference herein to a second cancer therapy typically refers to an alternative therapy to the first cancer therapy. The second cancer therapy can be but is not always a standard of care therapy. Thus, the present invention contemplates having patients in the experimental arm of a clinical trial to be switched to standard of care therapy if the experimental therapy is not efficacious, while still staying within the experimental arm, not the control arm, of the clinical trial. Alternatively, they could first be administered SOC and rapidly switched if it was not being efficacious.

In one aspect of the method of performing a clinical trial, the clinical trial further comprises administering a standard of care (SOC) therapy regimen to a control group of patients having the cancer. In some aspects of the invention, patients are randomized between the first group of patients (i.e., the experimental or test arm) and the control group of patients.

Another aspect, is to obtain regulatory approval, insurance coverage, payment or reimbursement comprising comparing the fraction of patients who will benefit from the first group/arm to the fraction that benefits from the SOC.

The first cancer therapy regimen includes but is not limited to immunotherapy, therapy using an antibody, adoptive T cell therapy, chimeric antigen receptor (CAR) T cell therapy, therapy using an antibody-drug conjugate, a cytokine therapy, therapy using a cancer vaccine, therapy using a checkpoint inhibitor, radiation therapy, surgery, therapy using a chemotherapeutic agent, a therapy using a targeted therapy, a therapy using an enzyme inhibitor and combinations thereof. In one aspect, the first cancer therapy regimen comprises administration of a drug that failed one or more prior Pivotal clinical trials. It is known to those of skill in the art that Phase III clinical trials are a subset of Pivotal clinical trials, also referred to as Pivotal trials. In one aspect, the first cancer therapy regimen comprises administration of a drug that previously failed a Pivotal trial and wherein the second regimen includes administration of SOC therapy. In another aspect, the first cancer therapy regimen comprises administering a drug that has not been in a pivotal trial and wherein the drug is the first drug in the first cancer therapy regimen. In another aspect, the first cancer therapy regimen comprises administering SOC and the second therapy comprises a NCD. As used herein, reference to a Pivotal clinical trial means a Phase III clinical trial or a Phase II clinical trial (if in the United States, the protocol for that Phase II Trial shall have been reviewed by the FDA under its current Special Protocol Assessment Guidelines (or equivalent guidelines issued in the future), and any comments from the FDA on that protocol are incorporated in the final protocol for that Phase II Trial or are resolved to the FDA’s satisfaction as evidenced by further written communications from the FDA; or if in Europe, a process with a comparable result - acceptance of a Phase II Trial protocol as “potentially pivotal” -- has occurred with the EMA or other Regulatory Authorities in the EU; or (iii) based on the results of that Phase II Trial, either the FDA or the EMA has determined that the Phase II Trial can be considered as a pivotal clinical trial for purposes of obtaining Regulatory Approval).

The biological sample provided in the methods disclosed herein is not particularly limited, provided it is known to potentially contain a nucleic acid marker indicative of cancer burden. In specific embodiments, the biological sample is selected from the group consisting of whole blood, blood plasma, blood serum, saliva, urine, cerebrospinal fluid, sputum, broncho-alveolar lavage, bile, stool, pleural effusion, lymphatic fluid, cyst fluid, stool, uterine lavage, vaginal fluids, ascites, and combinations thereof.

According to the present invention, in contrast to conventional methods such as imaging-based methods, the therapeutic efficacy of a cancer therapy regimen can advantageously be determined at an early stage after onset of said cancer therapy regimen. Accordingly, in preferred embodiments, of the methods disclosed herein, the biological sample is obtained from the patient(s) within 1 hour, within 2 hours, within 3 hours, within 4 hours, within 5 hours, within 6 hours, within 8 hours, within 10 hours, within 12 hours, within 15 hours, within 18 hours, within 21 hours, within 1 day, within 2 days, within 3 days, within 4 days, within 5 days, within 6 day, within 7 days, within 8 days, within 9 days, within 10 days, within 11 days, within 12 days, within 13 days, within 14 days, within 3 weeks, within 4 weeks, within 5 weeks, within 6 weeks, within 7 weeks, or within 8 weeks after onset of the first cancer therapy regimen or after a previous sample was obtained. In a further aspect, one or more biological samples are obtained from the patients prior to the onset of the first cancer therapy regimen.

In specific embodiments, the methods disclosed herein are repeated for at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30 times during the first cancer therapy regimen, e.g., for any number of n times, n being an integer 1 ≤ n ≤ 120, over the course of the cancer therapy regimen. Further, said methods can be performed 1, 2, 3 or more times prior to the onset of the cancer therapy regimen, e.g., to determine a baseline cancer burden or a baseline rate of change of cancer burden.

In specific embodiments, the methods disclosed herein are repeated every day, every other day, every third day, twice a week, weekly, every 10 to 14 days, every two weeks, every three weeks, every four weeks or every eight weeks during the first cancer therapy regimen.

The first cancer therapy regimen is identified as being efficacious when two or more measurements of the nucleic acid marker do not indicate a higher cancer burden or higher growth rate of cancer burden, and wherein the first cancer therapy regimen is identified as not being efficacious when two or more measurements of the nucleic acid marker indicate a higher cancer burden or higher growth rate of cancer burden. A higher cancer burden or higher growth of cancer burden is determined to be when the nucleic acid marker is at least 50%, 40%, 30%, 20%, 10%, or 5%, higher than a measurement of (i), (ii), (iii) or (iv) (i.e., one or more prior measurements of the nucleic acid marker in that patient after the onset of the first cancer therapy regimen ((i) and/or (iii)) or one or more measurements of said nucleic acid marker in that patient prior to the onset of the first cancer therapy regimen ((ii) and/or (iv))). In another aspect, the identification of the first cancer therapy as being efficacious or not efficacious is based on a trend or a change in trend of two or more measurements of the nucleic acid marker. Various methods to determine a change in the trend or slope and time at which the change occurs are know in the art (Skates, S. J., et al. Early Detection of Ovarian Cancer using the Risk of Ovarian Cancer Algorithm with Frequent CA125 Testing in Women at Increased Familial Risk-Combined Results from Two Screening Trails; Clin Cancer Res. 2017 July 15; 23(14):3628-3637).

In specific embodiments, the cancer burden of adjacent repetitions is compared, or in some averaged, to identify the cancer therapy regimen as being efficient or as not being efficient. A rate of change of cancer burden can be determined from the cancer burden of respective repetitions, e.g., of adjacent repetitions. “Stable cancer burden” can mean that the amount of cancer as determined by a nucleic acid marker is the same or that despite the cancer burden continuing to increase, there is no change in the growth rate of the cancer burden. Thus, a “stable cancer burden” can mean either the growth rate is the same as before, or the growth rate is zero or the cancer burden is unchanged.

In further specific embodiments, the patients have a cancer selected from the group consisting of a head and neck cancer, a central nervous system cancer, a lung cancer, a bronchial cancer, a mesothelioma, an esophageal cancer, a gastric cancer, a gall bladder cancer, a liver cancer, a pancreatic cancer, a melanoma, an ovarian cancer, a small intestine cancer, a colorectal cancer, a breast cancer, a kidney cancer, a renal pelvis cancer, a bladder cancer, a uterine cancer, a cervical cancer, a thyroid cancer, a muscle cancer, an endocrine cancer, a lymphoma, a bone marrow cancer, a leukemia, a myeloid dysplasia, an epithelial cancer, a cancer derived from endodermal tissue, a cancer derived from ectodermal tissue, a cancer derived from mesodermal tissue, and a prostate cancer.

In specific embodiments, a first cancer therapy regimen being identified as not being efficacious is a cancer therapy regimen allowing cancer progression in a patient, or cancer progression at an increased rate in a patient or a cancer therapy regimen allowing cancer hyperprogression in a patient.

The term “marker” as used herein relates to a measurable indicator of cancer burden. Accordingly, the term “nucleic acid marker” as used herein relates to a nucleic acid as a measurable indicator of cancer burden. In specific embodiments, the nucleic acid marker is a nucleic acid marker having a short half-life, e.g., wherein the half-life of the nucleic acid marker is less than 48 hours, less than 24 hours, less than 12 hours, or less than 6 hours.

In specific embodiments, the nucleic acid marker is a DNA marker, preferably a circulating short DNA marker, more preferably a cfDNA (cell free DNA) short marker, and most preferably a ctDNA (circulating tumor DNA) marker, i.e., a ctDNA that is measurably indicative of cancer burden. In one aspect, the DNA marker comprises a methylation status of said DNA. In still another aspect, the DNA marker comprises size distribution of said DNA. For example, the ratio of DNA pieces of about 50 base pairs to DNA pieces of about 166 base pairs can be used as a DNA marker. Further yet, the DNA marker comprises DNA sequence information. In another aspect, the DNA marker does not comprise DNA sequence information.

Methods of measuring respective nucleic acid markers are not particularly limited and are known in the art. Preferably, measuring said nucleic acid marker in said biological sample in the methods disclosed herein encompass the use of an electrode. Preferably, said electrode is a gold electrode or a graphene electrode, wherein a bare gold electrode is particularly preferred. Said bare gold electrodes can be a solid gold electrode, a screen-printed gold electrode, or a thin foil gold electrode. In further specific embodiments, measuring said nucleic acid marker in said biological sample encompasses the use of electrochemistry, preferably encompassing differential voltammetry, square wave voltammetry, or impedance measurements. Of note, in the methods disclosed herein, the nucleotide sequence of the nucleic acid used for the nucleic acid marker does not have to be known. Accordingly, measuring the nucleic acid marker in the biological sample does not require sequencing, mutation detection, arrays, methylation sequencing, bisulfite conversion, chemical conversion of a nucleic acid, next-generation sequencing (NGS), low-pass sequencing, bar coding, copy number alterations, PCR or amplification methods.

The subject in the methods according to the first aspect of the present invention can be any animal, preferably a mammal, more preferably a human. A patient may be a person, pet, or any animal being treated and the term “patient” is equivalent to the term “subject” as used herein.

Further in the method of performing a clinical trial, if the first cancer therapy regimen is identified as not being efficacious in a patient, the patient is administered a second cancer therapy regimen. In another aspect, the method further comprises administering a third cancer therapy regimen to a control group of patients having the cancer. In one aspect, the third cancer therapy comprises standard of care therapy for the cancer. In still another aspect, the second cancer therapy comprises standard of care therapy for the cancer. In yet another aspect, the method further comprises comparing the efficacy of treatments received by the first group of patients with the efficacy of treatments received by the control group of patients. Still another aspect, the first group of patients and the control group of patients have the same result on a pre-trial companion diagnostic test. The term “same result” is defined herein as similar enough result to categorize the patients with regard to the likelihood of receiving benefit from the proposed therapy.

Another embodiment relates to a method of performing an early-stage clinical trial. This method comprises administering a first dose level of a first cancer therapy regimen to a first group of patients; determining the therapeutic efficacy of the dose level for patients in the first group of patients by a method, comprising measuring a nucleic acid marker of cancer burden in a biological sample from the patients; wherein the first cancer therapy regimen is identified as being efficacious in a patient when the nucleic acid marker indicates a lower cancer burden or a stable cancer burden as compared to (i) one or more prior measurements of the nucleic acid marker in that patient after onset of the first cancer therapy regimen or (ii) one or more measurements of said nucleic acid marker in that patient prior to onset of the first cancer therapy regimen, and wherein the dose level is identified as not being efficacious in a patient when the nucleic acid marker indicates a higher cancer burden as compared to (iii) one or more prior measurements of the nucleic acid marker in that patient after onset of said cancer therapy regimen or (iv) one or more measurements of the nucleic acid marker in that patient prior to onset of the first cancer therapy regimen; and continuing to administer the first dose level to a patient when the first cancer therapy regimen is identified as being efficacious in the patient, and discontinuing the patient from the trial or escalating that patient to a higher dose level if the first dose level is identified as not being efficacious.

Another embodiment relates to a method of evaluating a hospice patient on a cancer therapy regimen. The term “hospice” means any time curative therapy or a therapy causing a remission is not anticipated. This method comprises administering a cancer therapy regimen to a hospice patient; determining the therapeutic efficacy of said cancer therapy regimen in the hospice patient by a method comprising measuring a nucleic acid marker of cancer burden in a biological sample from the patient; wherein the cancer therapy regimen is identified as being efficacious when the nucleic acid marker indicates a lower cancer burden or a stable cancer burden as compared to (i) one or more prior measurements of the nucleic acid marker after onset of the cancer therapy regimen, or (ii) one or more measurements of said nucleic acid marker prior to onset of the cancer therapy regimen, and wherein the cancer therapy regimen is identified as not being efficacious when the nucleic acid marker indicates a higher cancer burden as compared to (iii) one or more prior measurements of the nucleic acid marker after onset of said cancer therapy regimen, or (iv) one or more measurements of the nucleic acid marker prior to onset of the first cancer therapy regimen; and continuing to administer the cancer therapy regimen to the patient when the cancer therapy regimen is identified as being efficacious in the patient, and discontinuing the cancer therapy regimen when the cancer therapy regimen is identified as not being efficacious in the patient. Thus, in this embodiment, a cancer therapy regimen can be evaluated on a hospice patient and if the regimen is not efficacious, it will be discontinued without switching the hospice patient to an alternative therapy, such as standard of care. Such patients would be administered palliative care. In these patients, the cancer therapy regimen is not causing a benefit and can have high negative side effect profiles.

Another embodiment relates to a method of evaluating a cancer patient to be given a cancer drug therapy that might cause hyperprogression. This method comprises determining the rate of growth of the patient’s cancer by performing at least 2 measurements of circulating DNA prior to beginning of the therapy; determining the rate of growth of the patient’s cancer by performing at least 1 measurement of circulating DNA after a first measurement after beginning the therapy; and continuing to administer the therapy if the rate of growth has not increased or discontinuing the therapy if the growth rate has increased. Reference to the term “beginning of therapy” means either prior to the beginning of therapy or shortly thereafter.

Another aspect, relates to a method of performing a clinical trial for a novel cancer therapy regimen, comprising the steps of: (a) forming a trial arm in which patients receive said novel cancer therapy regimen and the therapeutic efficacy of said novel cancer therapy regimen in each patient is determined by the method according to the first aspect of the present invention, and (b) in case the novel cancer therapy regimen is identified as being efficient, the patient continues to receive the novel cancer therapy regimen, and in case the novel cancer therapy regimen is identified as not being efficient, the patient is switched to a different cancer therapy regimen but remains in the same trial arm.

In specific embodiments, said different cancer therapy regimen can be the standard-of-care (SOC) cancer therapy regimen, a second novel cancer therapy regimen, or a combination of the first novel cancer therapy regimen with a further cancer therapy regimen. In further specific embodiments, the novel cancer therapy regimen comprises the use of 2, 3 or more therapy regimens in a sequence, wherein the timing of said sequence is dependent on the outcome of performing the method according to the first aspect of the present invention.

In yet further embodiments, the methods according to performing a clinical trial for a novel cancer therapy regimen can further comprise the step of (i) seeking regulatory approval of the novel cancer therapy regimen based on said clinical trial, (ii) determining the best cancer therapy regimen for future patients based on said clinical trial, (iii) altering the design of the clinical trial depending on the outcome of performing the method according to the first aspect of the present invention, (iv) altering the therapy plan for individual patients in the trial depending on the outcome of performing the method according to the first aspect of the present invention, and/or (v) determining what dose of the novel cancer therapy regimen a particular patient has the best response to depending on the outcome of performing the method according to the first aspect of the present invention.

In specific embodiments of the methods according to performing a clinical trial for a novel cancer therapy regimen, a companion diagnostic test specifies who is admitted to the trial or to some of the arms in the trial. In further specific embodiments of said methods, the clinical trial is designed to seek evidence that a drug which has failed one or more prior Pivotal trials can be successfully and beneficially used and/or can receive regulatory or payment approval. This often involves comparison to a SOC group/arm.

In yet another aspect, the present invention relates to a method for determining an optimal cancer therapy regimen for an individual subject receiving a first cancer therapy regimen, comprising the steps of determining the therapeutic efficacy of said first cancer therapy regimen in the subject by the methods according to the first aspect of the present invention, and in case the first cancer therapy regimen is identified as being efficient, the patient continues to receive the first cancer therapy regimen, and in case the first cancer therapy regimen is identified as not being efficient, the patient is switched to a different cancer therapy regimen. The different cancer therapy regimen is the standard-of-care (SOC) cancer therapy regimen, a second cancer therapy regimen, or a combination of the first cancer therapy regimen with a further cancer therapy regimen. This method further comprise the step of (i) altering the therapy plan for the subject depending on the outcome of performing the method according to the first aspect of the present invention, (ii) determining what dose of the cancer therapy regimen the subject has the best response to depending on the outcome of performing the method according to the first aspect of the present invention, (iii) determining if a cytotoxic therapy designed to kill rapidly dividing cells is appropriate to be added or substituted for the first cancer therapy regimen dependent on the outcome of performing the method according to the first aspect of the present invention, (iv) determining whether continuing the first cancer therapy regimen is cost effective dependent on the outcome of performing the method according to the first aspect of the present invention, and/or (v) adjusting pricing or billing for the first cancer treatment regimen dependent on the outcome of performing the method according to the first aspect of the present invention.

As disclosed herein, the use of one or more biological samples, such as blood, using markers that have a half-life of less than 48 hours, preferably less than 24 hours, and more preferred less than 12 hours, especially most preferred well under six hours, may be used to accurately determine the higher cancer burden or higher growth rate of cancer burden prior to therapy and the change in this growth rate. While an absolute increase in ctDNA (circulating tumor/cancer DNA) of 20% has been defined by some as molecular progression or molecular progressive disease (MPD), this invention has newly determined that a change in the slope of growth, as measured by any appropriate change in an appropriate measure of ctDNA indicated what is herein “Accelerating Progressive Disease” (APD). APD has not previously been used, the invention is using short half-life markers to determine any increase in rate of growth and using this to quickly stop the therapy associated with APD. All previous definitions of HPD require a massive increase in the rate of growth - typically two-fold. RECIST 1.1 criteria for progression by imaging of a 1 cm lesion requires over a 300% increase in volume, for a 2 cm lesion RECIST 1.1 criteria is a 200% increase.

While inaccuracies or biologic variation, dependent on the particular ctDNA and its measurement may in some systems cause patients and physicians to require additional timepoint(s) and the change in the value to exceed a threshold to avoid being due to variation or error, it is herein revealed that any increase in the rate of growth is APD and that this is sufficient to result in a change in therapy.

There is some belief that the type of cancer and how malignant the cancer is may result in a different rate of release of ctDNA between patients. People may also have different clearance rates. However, in an individual patient the amount of ctDNA in the blood is a reliable indicator of the cancer growth and/or cancer burden and response to therapy. Total circulating cell free DNA (cfDNA) measured by some systems might include a contribution from both the host and the host reaction to the cancer, so that care must be taken to measure ctDNA (circulating tumor/cancer).

Herein is described a method of using certain measures of cfDNA as the definition of molecular HPD and APD and diagnosis of clinical HPD, APD and PD, it defines Growing Disease (GD) and establishes it as a criterion for changing therapy.

These biological sample collections (such as a blood sample) may be carried out in a clinically convenient manner such as just before a new round/infusion of therapy. In one embodiment this is done the day prior to next therapy (this term includes the next round of therapy), in another within 3 days prior to the next therapy and in another within a week of the next therapy). In a further embodiment this is done the day a patient visits, before therapy, and is used to determine if that therapy should be given.

Often the rate of change in this ctDNA is then determined, in one embodiment the change from adjacent time points is calculated. This change is used to determine if the current therapy is appropriate or should be changed. In another embodiment simply the amount measured compared to the previous amount is determined. If the amount is increased therapy is changed. While it is not required, the rate of change prior to the beginning of therapy is compared to the rate of change on therapy; this requires two blood samples before therapy begins. Changes in the amount and rate of ctDNA during the first week of cytotoxic or targeted therapy and 2 weeks for immunotherapy may reflect acute killing and a decrease in the cancer and may be used as a marker of therapy effectiveness. As rarely an acute increase due to cancer death is seen, a repeat test may be done. Decreases even during this period mark therapy benefit. In contrast after this initial period, increases, especially increase separated by a week or more mark the need for a different therapy.

This invention may be applied to any body fluid. In a preferred embodiment they are applied to plasma, serum, saliva, fingerstick blood or urine. In some embodiments of any of the methods disclosed herein, a biological sample obtained from the subject comprises blood, plasma, serum, urine, cerebrospinal fluid, saliva, sputum, broncho-alveolar lavage, bile, stool, pleural effusion, lymphatic fluid, cyst fluid, stool, uterine lavage, vaginal fluids, ascites, and combinations thereof.

When a less than optimal response to therapy is seen, one embodiment includes evaluating that response to see what additional drugs might be added or substituted. For example, in the case that the invention detects fast growing cancer a drug specifically targeted to kill rapidly growing cells, such as an anti-metabolite, including but not limited to a nucleic acid base analogue such as 5-FU might be added to the failing therapy or substituted for the failing therapy.

In some embodiments, the therapeutic intervention is selected from: a different immunotherapy, an antibody, adoptive T cell therapy, a chimeric antigen receptor (CAR) T cell therapy, an antibody-drug conjugate, a cytokine therapy, a cancer vaccine, a checkpoint inhibitor, radiation therapy, surgery, a chemotherapeutic agent, combinations thereof, or other therapies known in the art or that become known in the art.

Further, the present invention relates to a new trial methodology. This methodology will allow approval of temporally complex and variable protocols that involve a new drug. The schedule will be determined by the performance of the sub parts (various drugs) as actually measured in that specific person, during the trial. For the select people in which they perform best, the new drug will be the key agent, while for others SOC may be switched to thus optimizing therapy of the entire population, including people for whom the new drug is not effective. One unique feature is that this group, those for whom the new drug fails, also remains active in an experimental arm of the trial.

A major change in the design is that in addition to potential study arms including (i) just the new drug or new drug combination (this arm is included in some embodiments but not others) and (ii) a control trials - the SOC, it also includes (iii) an arm in which there is rapid switching from the new drug to the SOC (or another new drug) based on early determination if the new drug is working or not based on an Efficacy Defining Diagnostic (EdDx). The invention entails the use of an EdDx to define a therapy path that is distinct from any other path. The trial in effect becomes a trial of a variable therapy path including an EdDx. This differs from past trials in that it is no longer a trial of a drug (or drug combination) but of a protocol plan based on an EdDx. Approval of this plan is then sought.

This invention uses methods to determine in less than half the time generally required to reliably see change by imaging, if the cancer is being decreased by the therapy given. Methods of measuring circulating cancer DNA, (ctDNA) are known in the art and may be used in the context of the present invention. Many of them apply to cancers with specific mutations or containing specific genes, such as EGFR mutations, or HPV genes, or gene signatures or methylation signatures of genes, some are complex multicomponent assays. These, or other cancer measuring assays that are known or become known, are also advantageous embodiments of this invention. These assays are typically problematic in that they are too time consuming and take too long to report results or too expensive to be done repeatedly for the most advantageous frequent monitoring used in the preferred embodiment of EdDx, however they may be used under certain circumstances and are herein included.

There are a large number of known protein markers. Unfortunately, these are not as accurate a predictor of cancer size. Further they often have half-lives (t½) or weeks. ctDNA t½ is hours, so it accurately reflects current disease levels and is a preferred embodiment.

Circulating cancer DNA identified by a change in, methylation patterns, an epigenetic changes, is described in (i) Sina AA, Carrascosa LG, Liang Z, et al. Epigenetically reprogrammed methylation landscape drives the DNA selfassembly and serves as a universal cancer biomarker. Nat Commun. 2018;9(1):4915. Published 2018 Dec 4. doi:10.1038/s41467-018-07214-w, (ii) SinaAA, Lin TY, Vaidyanathan R, et al. Methylation dependent gold adsorption behavior identifies cancer derived extracellular vesicular DNA [published online ahead of print, 2020 Jun 12]. Nanoscale Horiz. 2020;10.1039/d0nh00258e. doi:10.1039/donh00258e, or (iii) WO 2020/077409 A1, or other methods known or to become known in the art for determining presence of DNA from cancer cells in a body fluid are preferred embodiments. Release of other materials including proteins, or vesicles, such as exosomes, or other materials from tumor may be used. The use of extracellular vesicles or exosomes (together or separately EVs) are a preferred embodiment. It is important that the marker used correlates with classic measures of tumor response such as partial response and complete response as defined by RECIST criteria, or if appropriate irRECIST criteria, however it is most notable that these tumor measures are monitored and detected early in the course of therapy. It has been established that circulating cancer DNA decreases following effective therapy within a period of four weeks or less.

For some cytostatic or immunotherapeutic treatments, the timeframe, both for imaging and for the cell free DNA or other early detection method is prolonged. Nevertheless, cell free DNA method can detect both decreases in tumor size and confirm that no increase in cancer burden is occurring, in less than half the time needed for imaging.

Sample steps that may be included in the method are: (step 1) Determine a new drug to be tested as part of the new treatment program. (step 2) Determine the standard of care (SOC) to which the new treatment plan will be compared. (step 3) As a key part of one experimental arm define that if the new drug does not produce the desired effect within a fixed relatively short period of time, typically 6 weeks and often shorter, patients will switch to SOC or switch to another care regimen being tested. (step 4) Give the first new drug to this experimental arm. (step 5) Soon after giving the new drug, typically less than half the time required for accurate imaging assessment, test for cancer response, in the preferred embodiments the test is one for cancer DNA circulating in the plasma, or EVSs, however any test known to correlate with cancer mass or survival may be used. In a preferred embodiment testing is done weekly or every 10-14 days. Other embodiments test from daily to monthly. In some embodiments testing is done daily for the first week, then less frequently. (step 6) If the EdDx assay shows the therapy has good potential of working the patient stays on that therapy (for some drugs or therapies this may be no increase in the circulating cancer DNA, for other agents, the criteria will be a decrease) typically within four weeks. Longer periods may be used in some embodiments. (step 7) If the patient has not benefited as indicated by the EdDx s/he is switched to either the SOC, or in some trials designs to a second drug being tested. (Unlike previous designs the patient does not stay on a drug until they show signs of progressive disease by imaging and/or clinical features, rather they are switched if they do not show clear evidence of benefit). Switching based on EdDx showing lack of benefit is another novel aspect introduced by this inventor. This contrasts with switching due to signs of failure. While EdDx evidence of failure may be used to define switching in a preferred embodiment switching is based on anything less than clear evidence of benefit. (step 8) As part of the predesigned plan testing and therapy the patient may be switched multiple times, each time an EdDx shows that the patient is not achieving the desired threshold a switch occurs. (step 9) The goal of this novel study design is to identify a treatment plan (i.e. a plan that includes switching from one drug to another based on the results of an EdDx) test that includes the new drug. This contrasts with previous designs which only test the effectiveness of one drug or one drug regimen (multiple drugs given contemporaneously) compared to a standard. (step 10) Thus, an embodiment is the approval of a EdDx based switching protocol or treatment plan. To date there has been no approval or known submission to the FDA or other regulatory bodies of such a protocol for approval. (step 11) The results of the treatment plan, including the new drug, the EdDx test, and the treatment that the patient is switched to are evaluated as a single arm or single therapy plan. An initial trial these results may be compared to historic controls as the SOC. In a typical randomized trial, they are compared to the standard of care, at times they may be compared to alternative treatment plans that include different drugs or sequences of drugs with the rapid switching defined by EdDx testing. In one preferred embodiment the SOC is given as part of the experimental arm and an EdDx is used to switch appropriate patients to a NCD.

In a preferred embodiment the mechanisms of action of the SOC and new drug are distinct. Typically, the new drug may be an immuno-oncology (IO) drug and the SOC may be a chemotherapeutic (Chemo) regimen.

Designing trials to include comprehensive treatment plans including rapid switching based on EdDx testing is a novel concept. It leads to a new plan for drug approval; historically drugs have been approved for specific indications, at one time it was based on the source of the tumor, more recently it is defined by not only the source of the tumor but previous therapies a person may have had, most recently it is been defined by the presence of certain mutations, or other biologic markers, that make a tumor susceptible to a certain therapy. These biologic markers are used before the drug is given; they are typically called “companion diagnostics”. When a drug is approved in combination with a companion diagnostic both are required to be used to determine whether a patient should get the drug. An EdDx has limited analogy to a companion diagnostic. A companion diagnostic is given prior to therapy to, on a statistical basis, predict benefit. In contrast, an EdDx is give after treatment to measure benefit.

This invention calls for the approval of a sequence of NCDs, further that sequence is defined by using a EdDx test given after the NCD is administered. The patient’s individual response to the therapy, as defined by the EdDx test is used to define therapy in the next period. Thus, this invention uniquely uses an in vivo, in homo sapiens, in persona, assay.

The use of this method has surprising and unexpected results. Potentially most importantly it allows new drugs to be approved as part of an EdDx based treatment plan that would have failed, or which have failed, to meet the criteria necessary to have been approved for that indication when used without such a plan. They may be NCDs that have already failed classic Phase III testing. It combines the benefit of both the new drug and SOC in a single new experimental arm.

Classically, if a patient shows progressive disease (PD) by imaging in trials they are removed from the trial, designated a failure according to the progression free survival metric and released from the trial. However, with this new protocol early EdDx testing may lead to a second, or even third or fourth defined therapy within the experimental trial arm.

Cytostatic, cytotoxic and immune therapies may all decrease cancer volume and decrease the circulating cancer DNA resulting in an EdDx detection of stabilization or cancer shrinkage. Cancer growth is associated with increased ctDNA or EVs, or other parameters measured by the EdDx. On occasion it is possible to detect acute killing of cancer by a transient increase an EdDx.

Traditionally patients are encouraged to stay on the trial protocol/drug unless toxicity is unacceptable or the have progressive disease defined by imaging, either result causes them to be a “failure” and removed from the trial. After this they may receive another therapy. In contrast this invention predefines they remain in the trial and switch to another therapy. In a preferred embodiment switching occurs based on lack of success rather than failure. It might be best explained as patients are permitted to remain on the new drug only if they show benefit. In effect the method selects those patients most likely to respond and leaves them on theNCD. Patients that are uncertain, less likely to respond, or unlikely to respond are all quickly transferred to SOC. When SOC is low risk and likely beneficial, cutoffs designed to maximize SOC transfer are typically preferred. When SOC is poor or dangerous less aggressive switching may be used.

In another embodiment the method is used to direct care near the end of life. Patient in hospice often receive anti-cancer therapy as it reduces symptoms if it reduces growth of the cancer. EdDx may be advantageously used to determine if a patient near the end of life should get a full course of drug. By monitoring with EdDx on a weekly basis, a therapy designed to reduce symptoms by halting cancer growth or causing some shrinkage can be evaluated. If the EdDx determines it is not being effective in it can be promptly discontinued.

In trials and in clinical practice cost effectiveness is often determined. Prior to this, the cost effectiveness of a new drug was ascertained by new drug only treatment. One embodiment of this invention is the cost effectiveness of a protocol involving the new drug, but also involving the EdDx and the other drug(s) defined by the trial is determined. This may be used in trials, in clinical practice, to define policy, and to determine reimbursement. One embodiment is to evaluate continued reimbursement for drugs based on EdDx criteria. Another embodiment is for variable pricing of drugs based on their meeting EdDx criteria, i.e., the patient specific billing for a drug with lower performance by an EdDx would be lower. For clarification if the drug did not meet certain criteria by the EdDx method a rebate or credit would be issued, or the drug would be billed at a lower price. Another embodiment is the drug would initially have a lower price but if it was being effective as measured by EdDx a supplemental bill would be issued.

As is typical with trials the inclusion criteria are carefully chosen. The trial designs herein presented can work in concert with a companion diagnostic. The companion diagnostic evaluates patients before the drug for eligibility while EdDx evaluates them while under therapy for effectiveness.

The principles of the invention as outlined above can be incorporated into Phase III trials and post marketing including Phase IV trials. They may also be used for drugs that have failed Phase III trials.

The following experimental results are provided for purposes of illustration and are not intended to limit the scope of the invention.

### EXAMPLES

### Example 1: A Cytotoxic Drug Targeting Cancer Shrinkage Trial Showed High Response Rate to SOC and New Drug.

Drug company PharmX (X) has developed a potential drug designated PX 101. X desires the drug to be approved for some use as soon as possible, as it is believed to be beneficial in patients with lung cancer. The outcome being sought is complete remission (CR) and partial remission (PR). The classic design would be one study arm receiving PX 101 and another study arm receiving SOC. PX 101 works by a different mechanism of action then SOC, little or no cross resistance occurs.

In this example, SOC results in 30% of the patients achieving CR and 30% achieving PR. 60% are judged by RECIST 1.1 criteria appropriate for this type of drug, to receive benefit. PX 101 in a classic standard single drug protocol achieved 25% CR and 25% PR, thus benefiting 10% fewer patients than SOC and would normally be rejected as a failed drug, assuming no major changes in quality of life and side effects. In some embodiments based on the in silico analysis this arm would not be run.

However, this study added an Efficacy Defining Diagnostic (EdDx) method design arm in accordance with the present invention. PX 101 was given for 3 weeks and a rest week was used prior to the next round. Just prior to the next round of therapy the amount of ctDNA and cancer EVs was determined by an assay known in the art for each of the 100 patients in the EdDx arm. This allowed a decision about whether patients were likely on their way to a CR or PR, or not. All patients not likely on their way to CR or PR, were, according to the EdDx predefined plan, transferred to SOC. The 25 patients on the way to PR and 25 patients on the way to CR continued on and achieved those goals. Of the total hundred patients this was 50% successes. Of the 50 people transferred to SOC, since one month delay in treatment has little effect (5 percentage points) on the SOC therapy, 25% achieved PR and 25% CR, slightly down from the 30% CR 30% PR, if SOC therapy had begun immediately. Thus, in total 25 of the 50 patients transitioned within the EdDX arm to SOC from PX 101 had a beneficial outcome. Thus, in the group assigned to EdDx plan, a total of 75 patients achieved benefit. Since 75% exceeds the SOC 60%, PX 101 in combination with an EdDx protocol and transition to SOC, resulted in an added benefit. This allowed the EdDx protocol to be approved and reimbursed.

### Example 2: A Drug Targeting Cancer Shrinkage by Preventing Cell Proliferation (Growth Factor Deprivation) While Cancer Cells Continue to Turnover. A Trial With High Response Rates.

The below table (Table 1) shows the results of a three-arm study with sample size of 200 in each study arm analogous to Example 1. This cytostatic drug, PX 102, is judged by the same RECIST criteria as cytotoxic drugs. Again the middle arm is often not run.

TABLE 1

Study Arm

| | | | |
|---|---|---|---|
| | SOC | PX 102 | EdDx done during first month. Switch from PX 102 to SOC if PX 102 not showing clear benefit within the first month |
| n (patients) | 200 | 200 | 200 |
| CR | 60 | 50 | 50 + 25 (50 pts switched, 50% of these benefit) |
| PR | 60 | 50 | 50 + 25 (50 pts switched, 50% benefit, half are above) |
| No benefit | 80 | 100 | 50 |
| Patient Result relative to SOC | | In this arm: 20 pts damaged as a result of not following SOC | In this arm: 30 pts benefited as a result of the EdDx protocol rules for switching from PX 102 to SOC |
| Drug Result | | SOC is better. PX 102 is rejected | EdDx Protocol is beneficial. PX 102 moves forward to approval, in combination with EdDx & SOC. |

pts: patients

Again, a drug that would have failed standard design was allowed to proceed to approval and reimbursement as part of the EdDx protocol.

### Example 3: Immunotherapy (IO) Trial in Which IO Causes Some Cures, Some HPD but Few Partial Responses.

IO trials often use irRECIST (immune-related Response Evaluation Criteria In Solid Tumors) criteria for determination of benefit by imaging as these have been shown to better reflect long term clinical benefit. In brief, irRESIST is based on the observation that stable disease correlates with long term survival benefit and thus is classified as beneficial. This is likely due to the fact that the immune system provides continuing long-term benefit and does not cause mutations that lead to even more aggressive cancer.

It is even more difficult to use imaging to predict long term benefit for patients on IO, for instance in view of said pseudo-progression caused by immune cells migrating into a cancer, which then cause swelling and an apparent increase in size by imaging. As mentioned before, this is a false positive signal of progression. Thus, repeated imaging at a subsequent time may be required to understand the significance of enlargement of a cancer on initial imaging. In fact, response criteria often require repeat imaging. Further, with immunotherapy, patient benefit likely results if there is a decrease or no change in the sum of the total body cancer burden, however much of this burden may be hidden in small lesions and not easy to quantify by imaging. Further, imaging using unidimensional measurement (the current standard), or bi-dimensional measurements, reflects but is not the same as volume measurements. There may be a factor of 8x difference. Nevertheless, the same principles as in the previous examples hold true. An earlier determination by EdDx molecular methods in accordance with the present invention, most especially measurement of circulating cancer DNA or EVs, can be advantageously used as a method to detect the benefit of an IO soon (within weeks) after the therapy is started. IO can cause accelerated growth of the cancer, in about 20% of the cases, thus it is critical to evaluate often and early to prevent iatrogenic harm during the IO portion of a rapid switching trial. A switch to chemotherapy during a phase of rapid growth is especially advantageous as chemo is especially effective at killing rapidly dividing cells. For IO the number of EdDx determinations and time a person on therapy who is not hyperprogressing or obviously progressing may be extended as cancer shrinkage may progress more slowly on IO than chemotherapy.

IO has unique advantages and disadvantages. At present it is virtually the only therapy for advanced cancer that can lead to long term, durable remissions or even cures. However, it does so in only a subset of patients and cancers. Even for the cancers it is approved to treat (the most responsive ones) in general, only one third of the patients have a response, even by irRECIST criteria. Sadly, only a third of those tend to be durable, but these cases are miraculous savings of otherwise, even with SOC therapy, disease that would be rapidly fatal. The goal of this study is to determine early who might be in this group and move all others to SOC to avoid hyperprogression and iatrogenic harm.

As described in the above examples, EdDx testing can be used to relatively quickly determine who is actually responding to an IO and thus who should be continued, and very importantly determining who is not responding to the IO, or hyperprogressing, and thus should be switched to SOC or another new therapy.

“Companion Diagnostics” (CDx) have been used to determine which patients are most likely to be responsive to an immunotherapy; these include expression of certain immune markers, including PD1 and/or PDL1 and other factors suggestive that the cancer may have high mutation rate, including sequencing or “microsatellite instability” (MSI high).

Unfortunately, these predictive tests are neither as sensitive nor as specific as needed to determine on a person-by-person basis who will respond and who will not. Thus, even companion diagnostic selected patients still, more often than not, fail the immunotherapy and as described above imaging can take months to determine whether failure is occurring. This is the term used for actually fast increase in cancer mass, not just tumor mass. These factors all indicate the importance of a EdDx test and rapidly switching therapies based on the results of that test. As noted in italics in the below table (Table 2), immunotherapy may result in a smaller number of patients who benefit but some cases of CR that experience extreme benefit thus presenting a treatment dilemma, the use or EdDx helps solve. Without EdDx IO 103 results in fewer patients benefiting, but some benefiting more. With EdDx more patients benefit compared to SOC and many more have CRs and long-term benefit.

TABLE 2

Study Arm

| | | | |
|---|---|---|---|
| | SOC chemo | IO103 alone | IO103 to SOC Chemo defined by EdDx. Pts with stable disease or decreasing disease by EdDx remain on IO |
| n (patients) | 200 | 200 | 200 |
| CR | 0 | 20 | 20 |
| PR | 100 | 20 | EdDx often indicated poorer than stable disease (n=120)]. These patients were switched to SOC. 40%¹ of patients transitioning based on EdDx showed benefit (48 pts). These 48 plus the above 20 resulted in 68 benefiting |
| Stable Disease with benefit | Not benefit | 40 | 40 pts show stable disease, on IO and remaining on IO with improved survival. |
| Pts who benefit | 100 | 80 | 108 |
| No benefit | 100 | 120 | 92 |
| Patient Result | | 20 additional pts damaged (no benefit) as a result of not following SOC however 20 CRs | 20 pts get CRs in this group compared to zero with SOC. 8 fewer persons with no benefit compared to SOC. |
| Drug Result | | 50% fewer pts benefit, however benefit can be longer. Creates clinical dilemma. Drug may or may not be approved. | EdDx Protocol is beneficial in all regards compared to IO or SOC alone. IO103 moves toward approval as part of EdDx program including switching to SOC |

¹ In standard of care 50% of patients benefit, 40% is used here to allow for any potential harm to the relatively short period they were on IO103 and not standard of care

While payers hesitated to pay for IO alone as the average benefit did not improve and IO was much more expensive than SOC, they gladly paid for EdDx as most patients switched dramatically reducing costs and some patients were cured, also dramatically decreasing costs. As pts with no benefit were reduced and cures experience IO was cost effective as well as approved by the FDA.

### Example 4: Phase I/II trial.

Phase I/II cancer trials are extremely problematic in that very few treated patients ever benefit. In fact, the problem is so severe that the ethics of performing such trials has been extensively discussed and questioned. With so few benefitting, the trials apparently violate the prohibition of experimenting on one person to benefit another, in this case future patients. The best justification is that they give people in the Phase I/II trial “hope”; however, such hope is for the most part a misrepresentation.

Most people are given doses of the drug being tested that are too low to have any chance of benefit. This is because drug dosing is begun at a level of about ⅓ to ⅕th the dose predicted to be safe by animal studies to avoid human specific toxicity that cannot be predicted. If the starting dose is determined from rodent toxicity, rather than larger animals, it is often ⅒ the anticipated dose. This caution is used in order to avoid causing side effects. Similarly, escalation is done cautiously and slowly so the total number of under-dosed subjects is high. A few lucky ones get potentially beneficial doses, but the escalation schedule moves on to give other doses that in the future will be found to be too high and cause excess toxicity. The odds of being in the Goldilocks’ position are so small that the overwhelming majority of patients are in the trial only to benefit others.

In this example, an EdDx protocol according to the present invention was used to allow rapid transfer to SOC of patients who receive doses too low to be beneficial. It improves patient care, as well as decreasing the ethical issue. Since information about toxicity is found from those on high doses, moving this group of low dose patients to SOC does not decrease the information on toxicity learned from the trial. In other studies of this type when no SOC was available the patients received a higher dose or another NCD.

Further if no sign of efficacy by EdDx, had been seen even at doses showing toxicity, this would have been informative to the sponsor and led to discontinuation of this drug. However, EdDx revealed that at 5-6x the starting dose a decrease in ctDNA was seen in one patient. This allowed increasing the number of patients given this potential beneficial dose from 3 to 6 and making that change for the escalating cohort. While standard escalation reveals almost no information on benefit, EdDx helps determine whether there was potential benefit and increases the number of patients on potentially beneficial doses, this allowed other examples of apparent efficacy. Further, in this example it was found that ctDNA (the EdDx measure used here) had a maximal response in patients at 5.5x the starting dose and appeared to taper off at higher doses. This led to a trial modification to start people on this dose and continuation of this promising drug candidate. Imaging alone was insensitive and did not show sufficient signs of efficacy to allow the program to continue. Thus, EdDx provided the sponsor important information that the drug may be effective.

“Adverse Events” (AE) include any negative event, the most severe being death. If an AE occurs, it must be determined, often with great difficulty and uncertainty, if it is a result of the treatment or due to the underlying disease. Transferring people whose disease was progressing by EdDx reduces this serious difficulty, as fewer progressing patients are on study and allowed cleaner determination of the toxic dose of the drug. Further, studying toxic effects in people known to have growing cancer on therapy is clearly unethical as well as unscientific.

Currently meeting imaging criteria for progressive disease by imaging results in removing someone for a trial, long after cancer growth and harm has been done. Imaging often requires a 200 to 300% increase in volume to identify progressive disease. Identifying likely progressive disease and switching earlier via EdDx is good medicine. It is harmful to the trial and sponsor, as well as the patient, to remain on drug if EdDx indicates progression. Put another way, EdDx decreases adverse events that are a result of progressive disease; this is another result that is helpful in the development of that drug. Again, early switching based on the blood level of circulating cancer DNA or EVs optimizes clinical trial design.

### Example 5: Phase I/IIa Trial

In this trial patients are accrued in groups of 3. Testing begins at 01 mg/kg. 5 dose groups show no toxicity, patients with signs of no benefit based on EdDx are transferred to another drug or SOC early, saving 15 people from on study progression. 2 dose groups (6 patients) get potentially beneficial doses. EdDx resulted in 3 remaining on study as they showed benefit and in early transfer of 3 patients as they did not. The latter 3 are saved from progression and side effects from the drug. 9 patients got doses that will eventually be found to be higher than optimal. 4 of these are switched rapidly to SOC based on the results of EdDx, these people are saved from the likely side effects of elevated doses. The sponsor sees fewer adverse events as a result of cancer progression. The sponsor experience is restricted to side effects in people who are potentially benefiting; this is more relevant information. However, in light of somewhat less experience with side effects, additional patients at the first level, and all additional levels, at which EdDx does not cause all patients to switch, are added. This results not only in additional patients who provide information on side effects, it provides information on efficacy at doses likely to be beneficial. Second, it increases accrual at levels that are potentially beneficial so more patients may benefit, and the sponsor may see more responses thus getting more early data on efficacy. The exact schedule of increased accrual may vary from study to study. In drugs that pre-clinical evidence suggests may have activity well below their toxic level the sponsor may elect to dramatically increase accrual when EdDx switching based on lack of benefit slows or stops, i.e., at early signs of efficacy. In this example when the 3 that remain on study were first found, an additional 6 were accrued at this level. Further an additional 6 were accrued at the next 2 higher levels resulting in an additional 18 patients receiving potentially beneficial doses. No extras were accrued at the highest level since toxicity was evident. These additional patients were provided the chance of benefit at a potentially acceptable dose. Of course, those not receiving benefit were rapidly switched to SOC or another drug based on the EdDx test. Nevertheless, 18 more people provided evidence of initial toxicity and benefit. The 9 that were not switched provided evidence regarding longer term side effects and benefits. Thus, the use of this EdDx design, including both switching EdDx non-responders, and adding additional patients once EdDx provides evidence of potential response provides much more information and hence is labeled, a Phase I/IIa trial. EdDx may also be used to determine what dose a patient has the best response to. It is well known in medicine that optimum therapy often requires titration/adjustment based on that person’s response individual. As this design reduces toxicity, increases efficacy signals and increases the chance of benefit it is not only more ethical and cost effective, it is more attractive to patients and improves accrual.

### Example 6: A Phase IIIc Trial

Phase III trials are typically definitive trials done after Phase II suggests an optimal dose and likely benefit. Sometimes they unexpectedly fail. This represents a major loss for the sponsor, and potentially for patients if there is a way that the new drug could be beneficial. In this example, a Drug FD202 had failed clinical trials being no better than the SOC, more expensive, and more difficult to administer. It had a different mechanism of action (MOA) and no cross resistance to SOC. The sponsor took this failed drug and used it in an EdDx design in accordance with the present invention. 200 patients were planned to be accrued into 3 arms (i) SOC, (ii) FD202 and (iii) EdDx FD202 to SOC. Arm (ii) was rejected as unethical and unneeded since it was already known that FD202 produced no better results than SOC. To facilitate understanding of the results the in silico results and benefits of the rejected arm 2 are presented in italics below in Table 3.

TABLE 3

| | | | |
|---|---|---|---|
| | SOC | FD202 (projected) | EdDx done during first month. Switch from FD202 to SOC if EdDx did not showing clear benefit within the first month |
| n (patients) | 200 | 200 | 200 |
| CR | 60 | 60 | 60 + 20 (80 pts switched, 50% of these benefit) |
| PR | 60 | 60 | 60 + 20 (80 pts switched, 50% benefit, half are above) |
| No benefit | 80 | 80 | 40 |
| Patient Result relative to SOC | | No benefit over SOC | In this arm: 40 more pts benefited as a result of the EdDx protocol rules for switching from FD202 to SOC |
| Drug Result | | FD202 is rejected as not commercially feasible | EdDx Protocol is beneficial. FD202 moves forward to approval and successful marketing reducing the failure rate by 50%, when used in combination with EdDx & SOC. |

This is defined as a Phase IIIc™ trial. It is a novel design not previously used.

Again approval, reimbursement and increased enthusiasm from doctors and patients was seen. Even in examples where the improvement is smaller EdDX improves chances of a beneficial outcome.

### Example 7: Testing an EdDx Protocol for Two Novel Drugs

A drug company had two new drugs that work by different mechanisms evaluated “in silico” in this trial. Before this trial there was concern that each individually might not exceed the SOC sufficiently in a small clinical trial to be approved. They project the benefit seen in italics below in Table 4 for drugs R & L. Rather than testing them separately they tested them together based on EdDx rules resulting in the right most column of the below table. The arms not in italics below were performed, as above the italics arm was rejected as not necessary and not ethical given potential side effects.

TABLE 4

| | | | |
|---|---|---|---|
| | SOC | R or L Separately (projected) | EdDx done during first month. Switch from R to L if EdDx did not showing clear benefit within the first month |
| n (patients) | 100 | 100 | 100 |
| CR | 35 | 35 | 35 + 9 (30 pts switched, 60% of these benefit) |
| PR | 35 | 35 | 35+ 9 (30 pts switched, 60% benefit, half are above) |
| No benefit | 30 | 30 | 12 |
| Patient Result relative to SOC | | No benefit over SOC | In this arm: 18 more pts benefited as a result of the EdDx protocol rules for switching from R to L |
| Drug Result | | Both R & L rejected as not commercially feasible | EdDx Protocol is beneficial. Both R & L move forward (together) to approval and successful marketing as they reduce the no benefit rate by 60%, when used in combination. |

In addition, the Sponsor gained information from the EdDx about the initial success rate of R alone when used first. In another in silico example, the sponsor subdivided the EdDx group so that 50% got R first, and 50% received L first, which provided information about use as single agents. The sponsor had considered given R & L simultaneously, but this was rejected as they were both myelotoxic and thus dangerous to combine simultaneously. This EdDx provides a mechanism for using two drugs that cannot be used simultaneously in a single trial and procedure.

In analogous examples, R & L might have been drugs never before in a pivotal trial or drugs that each had previous failed Phase III trials since they were no better than SOC. While not shown in the table, all patients failing EdDx while on drug L were moved to SOC, thus further improving outcome. Most importantly the results of the EdDx test done on R and on L indicate the activity of each drug. The EdDx method of showing benefit is beneficial to the sponsors and provides additional information useful to regulators. When novel drugs are used together simultaneously it may be difficult to know whether each has activity. The design described here allows data concerning the efficacy of each drug, as a single agent, to be obtained in a trial that tests both agents and gets patients the benefits of both. This procedure thus was valuable in defining both R & L are beneficial. In other examples when new CRs or PRs resulted from switching to “L” and there was not a combined toxicity issue, subsequent studies combining L with R simultaneously were suggested. The above initial trials also gave information about toxicity that can help design a trial given the drugs simultaneously.

### Example 8: Adaptive Therapy

The above examples focused on drug therapy procedures that target curing patients and if not, achieving PRs. In this context, it has been shown in silico and in animals that it improved survival to give therapy that maintained a cancer at a certain size but that did not aim to cure the cancer. This process of maintaining the tumor at the same level was labeled “Adaptive Therapy”. It was demonstrated that this increased survival time in animals. A related finding was the discovery that a primary tumor could inhibit the growth of metastatic lesions. This again suggests keeping cancer at a constant volume might be more beneficial than attempting to cure in situations where it is known that cure is unlikely and fast growing recurrences are likely. A constant volume tumor is defined as a benign tumor. What makes tumors dangerous is that they expand locally and expand at a distant site (metastasize).

Adapting these potentially exciting finding to patients has been difficult in large part because it is difficult to frequently measure tumor volume and adjust therapy. In this in silico/in cogitation example an EdDx method in accordance with the present invention is used for frequent monitoring of EVs or ctDNA, to maintain a constant level without attempting to eliminate it. This resulted in extending the survival and in less toxicity from boluses of classic anti-cancer therapy, targeted to reduce cancer size.

EdDx can be used to time changes in the types of therapy and/or the amount of therapy to maintain a constant size tumor. This may be especially advantageously used along with new therapies that are believed to prevent metastases. This procedure may also be especially beneficially used in cancer such as head and neck. Once these recur it is highly unlikely that they will be cured. However, these cancers grow by local extension that cause high morbidity and ultimately mortality. Because they infiltrate an expanding complex manner, quantifying the total size of the cancer may be difficult, even if some of it is visible. This and other cases where local invasion rather distant metastases is the greatest concern are places adaptive therapy may be especially advantageously used EdDx may be used to quantify the amount of cancer and adjust therapy.

### Example 9: Examining a Preferred EdDx Method for Performance

cfDNA from normal and a cancer patient was obtained and analyzed for electrode binding as described in Sina et al 2018. A dilution series was made. The breast cancer pt cfDNA was tested at 100%, 50%, 20%, 10% and 5% in all cases the total cfDNA was held constant by adding cfDNA from a normal donor. The 0% reading is 100% normal donor and 0% breast cancer cfDNA. n = 4 @ 0%, n=5 at other %.

TABLE 5

Detection of Differences in Cancer DNA Concentration by aiElectrode

| | | | | | | |
|---|---|---|---|---|---|---|
| % patient DNA | 0% | 5% | 10% | 20% | 50% | 100% |
| Mean | 4.38 | 4.95 | 10.52 | 15.46 | 18.61 | 27.61 |
| std dev | 0.10 | 0.07 | 0.39 | 0.24 | 0.66 | 1.58 |
| CV | 2.2% | 1.5% | 3.7% | 1.5% | 3.6% | 5.7% |
| t-test p= | | 0.00005 | 0.0000017 | 0.0000001 | 0.00008 | 0.00002 |

LoB = 4.48 LoD = 4.49 LoB + 3 = 4.69

The low coefficient of variation (CV) show that the test is highly accurate and capable of easily distinguishing even a 5-10% change in ctDNA and thus cancer burden. The p values are for separation from the adjacent lower concentration of ctDNA. The extraordinarily low p values again indicate this is a quantitative method that easily distinguishes small changes in amount of cancer DNA.

### Example 10 Aggressive Switching EdDx Trial Design

Patients doing poorly on test Drug are switched to SOC with different MOA Only those clearly benefiting by EdDx testing remain on New Drug. This example includes conservative assumptions of effectiveness.

TABLE 6

Study Arm

| | | | |
|---|---|---|---|
| | SOC | New Drug Alone | Rapid Switching Arm EdDx EdDx done during first month. Switch from New Drug to SOC if New Drug is not showing benefit within the first month |
| n (patients) | 200 | 200 | 200 |
| CR or PR | 120 (60% response rate) | 100 (50% response rate) | 90 respond to New Drug (conservatively assumes some that would have benefited from New Drug are switched). 110 pts switched to SOC; 50% of these benefit (conservatively assume 50% response rate). 90 + 55 = 145 in total respond, 72.5% response rate in this arm |
| No benefit | 80 | 100 | 55 |
| Patient Result relative to SOC | | 20 pts damaged compared to SOC. | In this arm: 55 additional pts benefited compared to new drug alone and 25 benefited compared to SOC. |
| Drug Result | | SOC is better. New Drug is rejected | EdDx Protocol is beneficial. New Drug moves forward to approval, as part of combination with ctDNA assessment. |

ai-TRIAL Design:

Study with high response rates. Patients doing poorly on test Drug are switched to SOC with different MOA, assuming effective drugs and no change in effectiveness due to switching.

TABLE 7

Study Arm

| | | | |
|---|---|---|---|
| | SOC | New Drug | EdDx (CtDNA Diagnostic During Treatment) done during first month. Switch from New Drug to SOC if New Drug is not showing clear benefit within the first month. In this case EdDx was a urine test for ctDNA. In other examples it was a blood test. |
| n (patients) | 200 | 200 | 200 |
| CR or PR | 120 (60% response rate) | 100 (50% response rate) | 100 on New Drug respond. 100 pts switched to SOC; 60% (60) of these benefit (assume the same 60% response rate). 100 + 60 = 160, an 80% response rate in this arm |
| No benefit | 80 | 100 | 40 |
| Patient Result relative to SOC | | 20 pts damaged compared to SOC. | In this arm: 60 additional pts benefited compared to new drug alone and 40 additional pts benefited compared to SOC. |
| Drug Result | | SOC is better. New Drug is rejected | EdDx Protocol is beneficial. New Drug moves forward to approval, as part of combination with ctDNA |

This results in benefits regarding approval, reimbursement and frequency of use.

### Example 11

Later Line Low Response Rates of benefit – Assumption each drug less effective in switching paradigm. Aggressive Switching, only those clearly benefiting remain on New Drug.

TABLE 8

Study Arm

| | | | |
|---|---|---|---|
| | Standard | New Drug Alone | Rapid Switching Arm EdDx |
| | SOC | New Drug | EdDx (CtDNA Diagnostic During Treatment) done during first month. Switch from New Drug to SOC if New Drug is not showing benefit within the first month |
| n (patients) | 200 | 200 | 200 |
| CR or PR | 30 (15% response rate) | 25 (12.5% response rate) | 20 responders on New Drug (conservatively assumes some that would have eventually benefited from New Drug if not switched). 180 pts switched to SOC; 18 of these benefit (conservatively assume 10% response rate). 20 + 18 = 38, a total of 19% |
| No benefit | 170 | 175 | 162 |
| Patient Result relative to SOC | | 5 pts damaged as a result of not following SOC | In this arm: 18 additional pts benefited compared to new drug alone and 8 additional pts benefited compared to SOC. |
| Drug Result | | SOC is better. New Drug is rejected | EdDx Protocol is beneficial. New Drug moves forward to approval, as part of combination with ctDNA assessment. |

This results in benefits regarding approval, reimbursement and frequency of use.

### Example 12

Later Line with Low Response Rates. Conservative Assumption – SOC works less well if delayed. Conservative Switching – only those clearly not benefiting (i.e., with increasing growth) are switched, some who don’t benefit remain on IO.

TABLE 9

Study Arm

| | | | |
|---|---|---|---|
| | Standard | New Drug Alone | Rapid Switching Arm EdDx |
| | SOC | New Drug | EdDx (CtDNA Diagnostic During Treatment) done during first month. Switch from New Drug to SOC if New Drug is not showing benefit within the first month |
| n (patients) | 200 | 200 | 200 |
| CR or PR | 30 (15% response rate) | 25 (12.5% response rate) | 25 New Drug responders and 15 ultimately non-New Drug responders (had no change at time of switching decision) remain on IO. (All New Drug benefit captured as switching rule is conservative.) 160 pts switched to SOC; 16 of these benefit (conservatively assume 10% response rate). 25 + 16 = 41, a total response rate of 20.5% |
| No benefit | 170 | 175 | 159 |
| Patient Result relative to SOC | | 5 pts harmed compared to SOC | In this arm: 16 additional pts benefited compared to new drug alone and 11 additional pts benefited compared to SOC. |
| Drug Result | | SOC is better. New Drug is rejected | EdDx Protocol is beneficial. New Drug moves forward to approval, as part of combination with ctDNA assessment. |

While various embodiments of the present invention have been described in detail, it is apparent that modifications and adaptations of those embodiments will occur to those skilled in the art. It is to be expressly understood, however, that such modifications and adaptations are within the scope of the present invention, as set forth in the following exemplary claims.

What is claimed is:

1. A method of performing a clinical trial for a cancer therapy regimen, comprising the steps of
(a) administering a first cancer therapy regimen to a first group of patients having a cancer;
(b) determining a therapeutic efficacy of the first cancer therapy regimen for patients in the first group of patients by a method, comprising measuring a nucleic acid marker of cancer burden in a biological sample from the group of patients,
wherein the first cancer therapy regimen is identified as being efficacious in a patient of the group of patients when the nucleic acid marker indicates a lower cancer burden or a stable cancer burden as compared to
(i) one or more prier-measurements of the nucleic acid marker in the patient after onset of the first cancer therapy regimen; or
(ii) one or more measurements of the nucleic acid marker in the patient prior to onset of the first cancer therapy regimen,
and wherein the first cancer therapy regimen is identified as not being efficacious in the patient when the nucleic acid marker indicates a higher cancer burden as compared to
(iii) one or more measurements of the nucleic acid marker in the patient after onset of the cancer therapy regimen, or
(iv) one or more measurements of the nucleic acid marker in the patient prior to onset of the first cancer therapy regimen;
(c) continuing to administer the first cancer therapy regimen to the patient when the first cancer therapy regimen is identified as being efficacious in the patient, and discontinuing the first cancer therapy regimen and beginning to administer a second cancer therapy regimen to the patient when the first cancer therapy regimen is identified as not being efficacious in the patient, and retaining the patient receiving the second cancer therapy regimen in the first group of patients.

2. (canceled)

3. (canceled)

4. The method of claim 1, wherein the biological sample is obtained from the patients within 5 weeks, after onset of the first cancer therapy regimen, or after the previous sample was obtained.

5. (canceled)

6. (canceled)

7. The method of claim 1, wherein steps (b) and (c) are repeated every day, every other day, every third day, twice a week, or weekly during the first cancer therapy regimen.

8. (canceled)

9. (canceled)

10. The method of claim 1, wherein the identification of the first cancer therapy as being efficacious or not efficacious is based on a trend or a change in trend of two or more measurements of the nucleic acid marker.

11. (canceled)

12. The method of claim 1, wherein a first cancer therapy regimen identified as not being efficacious allows cancer progression in the patient, cancer progression at an increased rate in the patient, or allows cancer hyperprogression in the patient.

13. (canceled)

14. (canceled)

15. The method of claim 1, wherein the nucleic acid marker is a DNA marker.

16. The method of claim 15, wherein the DNA marker is a circulating DNA.

17. The method of claim 16, wherein the circulating DNA is a circulating tumor DNA (ctDNA).

18. The method of claim 15, wherein the DNA marker comprises a methylation status of the DNA marker.

19. The method of claim 15, wherein the DNA marker comprises size distribution of the DNA marker.

20. (canceled)

21. The method of claim 15, wherein the DNA marker does not comprise DNA sequence information.

22. The method of claim 1, wherein measuring the nucleic acid marker in the biological sample in step (b) comprises using an electrode.

23. The method of claim 22, wherein the electrode is a gold electrode, a bare gold electrode, or a graphene electrode.

24. (canceled)

25. The method of claim 23, wherein the bare gold electrode is a solid gold electrode, a screen-printed gold electrode, or a thin film gold electrode.

26. The method of claim 1, wherein measuring the nucleic acid marker in the biological sample in step (b) comprises using electrochemistry.

27. The method of claim 26, wherein electrochemistry comprises differential voltammetry, square wave voltammetry, impedance measurements, or a combination thereof.

28. (canceled)

29. The method of claim 1, wherein the method of performing the clinical trial further comprises administering a standard of care therapy regimen to a control group of patients having the cancer.

30. (canceled)

31. The method of claim 1, further comprising administering a third cancer therapy regimen to a control group of patients having the cancer, wherein the third cancer therapy comprises standard of care therapy for the cancer.

32. (canceled)

33. (canceled)

34. (canceled)

35. The method of claim 31, wherein the first group of patients and the control group of patients had the same result on a pre-trial companion diagnostic test, wherein the pre-trial companion diagnostic test is given to the patients prior to the patient receiving therapy in order to predict a benefit of the first cancer therapy.

36. (canceled)

37. (canceled)

38. (canceled)

39. A method of performing an early stage clinical trial comprising the steps of:
(a) administering a first dose level of a first cancer therapy regimen to a first group of patients;
(b) determining the therapeutic efficacy of the first dose level for patients in the first group of patients by a method, comprising measuring a nucleic acid marker of cancer burden in a biological sample from the first group of patients;
wherein the first dose level is identified as being efficacious in a patient in the first group of patients when the nucleic acid marker indicates a lower cancer burden or a stable cancer burden as compared to
(i) one or more prior measurements of the nucleic acid marker in the patient after onset of the first cancer therapy regimen, or
(ii) one or more measurements of the nucleic acid marker in the patient prior to onset of the first cancer therapy regimen; and
wherein the first dose level is identified as being not efficacious in a patient when the nucleic acid marker indicates a higher cancer burden as compared to
(iii) one or more prior measurements of the nucleic acid marker in the patient after onset of said cancer therapy regimen; or
(iv) one or more measurements of the nucleic acid marker in the patient prior to onset of the first cancer therapy regimen; and
(c) continuing to administer the first dose level to the patient when the first cancer therapy regimen is identified as being efficacious in the patient, and discontinuing the patient from the early stage clinical trial, or escalating the patient to a second dose level of the first cancer therapy regimen if the first dose level is identified as not being efficacious, wherein the second dose level is higher than the first dose level.

40. (canceled)

41. (canceled)
